(19) United States
(12) Patent Application Publication
(10) Pub. No.: US 2003/0186405 A1
(43) Pub. Date: Oct. 2, 2003

(54) Micro/nano-embossing process and useful applications thereof

(75) Inventors: L. James Lee, Columbus, OH (US); Siyi Lai, Columbus, OH (US)

Correspondence Address: STANDLEY & GILCREST LLP, 495 METRO PLACE SOUTH, SUITE 210, DUBLIN, OH, 43017, US

(73) Assignee: The Ohio State University Research Foundation

(21) Appl. No.: 10/113,233

(22) Filed: Apr. 1, 2002

(51) Int. Cl. C12N 11/04; C12M 1/12; A61K 9/48; A01N 63/00; A01N 65/00; A61K 38/28; B01D 39/00; B01D 71/06; G03F 7/26; B28B 1/48; B29D 19/08; B29C 33/40; F25C 1/24; B28B 7/06; A23G 9/00; B29C 33/00

(52) U.S. Cl. 435/182; 435/297.4; 424/451; 424/93.1; 514/3; 210/500.27; 430/320; 430/322; 264/154; 264/219; 249/129; 249/187.1

(57) ABSTRACT

The present invention relates to a method of producing micro and nano-porous polymeric articles with well-defined pore structures.

### TECHNICAL FIELD OF THE INVENTION

This invention relates to a method of producing micro and nano-porous polymeric articles with well-defined pore structures. The porous articles can be used in a variety of applications, including micro-transfer molding applications and micro/nano-filtering applications. Such applications enable useful products in a variety of fields, including the inhalation and intravenous drug delivery and immunoprotection fields.

### BACKGROUND OF THE INVENTION

The ability to produce polymeric substrates with micro and nano-pores wherein the pore structures are well-defined and the arrangement, size, and shape of the pores is controlled is of great use in differing applications.

One application contemplated by the present invention includes micro-transfer molding. The present invention tailors the transfer-molding method to render it useful for making particles of a micron and submicron scale. Transfer molding is widely used in polymer processing. Transfer molded products include, for example, rubber o-rings, gaskets, encapsulated IC chips, and contact lenses. Commonly, heat and pressure are used to transfer polymer material from the transfer pot into the mold cavity via a “sprue,” or tube.

An area in which it is highly desirable to quickly and efficiently mold polymer-type material into micron and submicron-sized particles of particular shape is related to the field of inhalation drug therapy. Certain drugs, including peptides and proteins, are unable to withstand stomach and intestine enzymes, and therefore need to be directly administered into the bloodstream. One way of achieving this is through the lungs. As such, inhalation devices such as nebulizers, metered dose inhalers (MDI's), and dry particle inhalers (DPI's) attempt to provide a means for delivering drugs to lung alveoli. The alveoli structures in the lung permit mass transfer to the blood stream. However, most mass transfer occurs in the deepest recesses of the lung, where the alveoli are located most densely. The repeated bifurcation of lung passageways provide a tortuous duct system for the airflow to follow to reach these alveoli. See generally, A. L Adjei & P. K. Gupta, ed., Inhalation Delivery of Therapeutic Peptides and Proteins, 5, 185 (1997). Since the branched system creates complicated air flow patterns and a tortuous path, and since most of the passages are lined with fluid to capture and remove particles, most particulate matter is removed before reaching the alveoli.

In general, the ability of particles to reach the alveoli depends on the size and density of the particles. Large particles, for instance particles above 5 μm in diameter, typically encounter the air-passage walls before reaching the alveoli, as inertial effects tend to override airstream currents. Smaller particles, for instance particles below 1 μm in diameter, tend to agglomerate, making their effective diameter large, and thereby also tend to encounter air-passage walls prior to reaching the alveoli. See Robert F. Service, Drug Delivery Takes a Deep Breath, 277 Science 1199 (1997). One way to overcome this challenge is to make particles more aerodynamic, thereby increasing the ability of the particles to stay suspended in the airstream along its tortuous path to the alveoli.

The present invention further contemplates novel micro and nano-filtering, or sieving, devices and methods for making the same. The ability to repeatably control the arrangement, shape, and size of micro and nano-pores in a polymeric substrate allows the filtering devices of the present invention to be utilized in a variety of useful applications including applications related to the biomedical industry.

Examples of such filtering applications include cell-based delivery and immunoprotection devices. One such immunoprotection device is produced by a method wherein a container is formed to encompass immuno-active cells, for instance insulin-producing cells. One side of the container, for instance the bottom, has well-defined nano-pores, or nano-tubes, that restrict the flow of particles of a size greater than the effective diameter of the nano-pores, while permitting the flow of particles smaller than the effective diameter of the nano-pores. In this example, producing a container with a side containing nano-tubes ranging in effective diameter from about 10 nanometers to about 100 nanometers (preferably about 10 to about 30 nanometers) allows for an insulin-producing device. Insulin-producing cells are contained within the container and are not permitted to escape, as they are generally an order or magnitude greater than the nano-pores just described. In the same manner, material noxious to the cells such as bacteria, viruses, and antibody molecules is prevented from entering the container, as it is also generally at least an order of magnitude larger than the container's nano-pores. More importantly, salutary materials such as insulin, salts and sugars are permitted to flow into and out of the container, as these substances are generally an order of magnitude smaller than the nano-pores previously described.

One can easily imagine, then, that present invention enables one to tailor such filtering, or sieving, devices in relation to one's need based on the ability to control the size, shape, and arrangement of pores, or tubes, in a polymeric substrate on a nano-scale.

### SUMMARY OF THE INVENTION

An embodiment of the present invention concerns a polymeric plate containing a plurality of nano-tubes arranged in a predetermined manner. Each nano-tube has two openings, or apertures. Each nano-tube can comprise any tube-like structure wherein the effective diameter of at least one aperture is less than about 100 microns. The use of the term “tube” should not connote a limitation to vertical structures per se, but encompasses any structure generally having two apertures connected by a conduit, including conical, pyramidal, square, and rectangular conduits, and the like. Similarly, the use of the term “tube” encompasses a “pore” structure, in that the depth of the tube can generally be less than the effective diameter of the apertures. The aforementioned tube apertures may or may not be equal in area or shape.

In one embodiment of the present invention, particularly useful in immunoprotective devices, at least one of each tube's apertures has an effective diameter in the range from about 10 nanometers to about 100 nanometers (preferably about 10 to about 30 nanometers). In another embodiment of the present invention, the polymeric plate is any photocurable or thermoplastic polymer.

The polymeric plate of the present invention provides the basis for nano-filtering and micro-transfer molding devices. In a micro-transfer molding device, the polymeric plate serves as a “sprue plate” for channeling the moldable material into the mold cavities. In a filtering device, nanoparticles with an effective diameter greater than the effective diameter of each of the plate's nano-tubes are blocked from passing through the nano-tube while particles with smaller effective diameters are permitted to pass through the tube. As one can easily see, the control over the size, shape and arrangement of the nano-tubes determines the effective functionality of a “micro-sprue” plate. Also, as one can easily see, the control over the size and shape of each nano-tube aperture permits the nano-filters of the present invention to be highly effective at screening nano-particulates based on size and shape. Such control over the size and shape of each nano-tube is accomplished by the precise manufacture of a nano-member array, which array is generally used as a template for the nano-tubes, as generally described below.

A nano-member array generally consists of any array of projections that will permit the formation of nano-tubes of the desired size and shape. Generally, any method capable of forming micro or nano surface features in a substrate is suitable for forming such a nano-member array. An array with micro-sized features can be manufactured, for example, through photolithography or DRIE followed by electroplating. An array with nano-sized features can be manufactured, for example, through differential etching, self-assembly, x-ray lithography, EBL, AFM indentation, or surface machining with a sacrificial layer.

In one embodiment of the present invention, a nano-member array of conical projections is manufactured by differentially etching a fiber optic bundle. Another method for manufacturing a nano-member array of conical projections is through the anisotropic etching of silicon. Such processes can yield conical projections with tip widths less than about 100 nanometers, and optionally the tip widths can be less than about 10 nanometers. Such processes are well-known in the art, and described in, for example, T. H. Dam and P. Pantano, Review of Scientific Instrumentation, 70, 3982 (1999); S. Henry, D. V. McAllister, M. G. Allen and M. R. Prausnitz, Journal of Pharmaceutical Sciences, 87(8), 922 (1998).

In another embodiment of the present invention, a nano-member array of pyramidal projections is manufactured by indenting a PMMA substrate using a diamond-tipped AFM probe, followed by casting PDMS on the indented substrate, whereby the resulting cast PDMS plate contains pyramidal projections as defined by the pyramidal indentations of the PMMA casting substrate.

It is to be understood that the preceding examples are not intended to limit the geometry of the nano-member array projections of the present invention. Any geometry capable of being manufactured by the previously mentioned methods and their equivalents is within the scope of the present invention.

One embodiment of the present invention concerns a method for making a polymeric plate containing a plurality of nano-tubes through the use of a sacrificial layer. A starting material arrangement is obtained comprising a dimensionally stable support substrate—for instance silicon, glass, or teflon; a sacrificial layer on the support substrate; and a non-sacrificial layer on the sacrificial layer. An array of nano-members is then impressed through the non-sacrificial layer and into the sacrificial layer and the sacrificial layer is subsequently removed.

The sacrificial layer may be any material capable of being preferentially removed from the non-sacrificial layer, including any suitable soluble polymer.

The non-sacrificial layer may optionally be in precursor form prior to impressing an array of nano-members through it. The method then contemplates setting the non-sacrificial layer prior to the removal of the sacrificial layer. An embodiment of the precursor material comprises any relatively low viscosity polymeric or oligomeric material, including thermoplastic solutions and spin-coated photocurable resins.

In a particular embodiment of the present invention, the nano-member array comprises projections having size and shape capable of defining nano-tubes with effective diameters on either of their ends from about 10 nanometers to about 100 nanometers (preferably about 10 to about 30 nanometers), or otherwise capable of being effective in filtering noxious materials in immunoprotective devices.

A further embodiment of the method of making a nano-tube plate for use in micro-transfer molding or filtering devices involves the additional step of providing a patterned layer over the non-sacrificial layer. Such a layer can act as a material container, or “transfer pot,” in association with the non-sacrificial layer. The patterned layer may be achieved by any suitable process. In one particular embodiment of the invention, the patterned layer is achieved by, but not limited to, photolithography.

The present invention contemplates a method of making a polymeric plate containing a plurality of nano-tubes that does not involve the use of a sacrificial layer. Such method comprises obtaining a polymeric bulk material that is sufficiently impressionable to accept an array of nano-members; impressing an array of nano-members into the bulk material; setting the bulk material; removing the nano-member array; and cleaving the bulk material in such a way as to form a plate having a plurality of nano-tubes, wherein both ends of the tubes have apertures. That is, cleaving the material so as to leave substantially all the nano-tube ends open.

Particular embodiments of the polymeric bulk material can comprise a partially cured thermoset polymer, for instance PDMS, or a heated thermoplastic, for instance PMMA.

Another embodiment of the present invention comprises a polymeric container having a plurality of nano-tubes arranged in a predetermined manner in a portion of it, and a method for making such a container. The container defines an inner volume and the nano-tubes are arranged so as to permit the inner volume to be in fluid contact with the environment outside the container. The container is capable of being any size and shape as determined by the method of making the container described below, but in one embodiment the container defines a volume about 1 microliter, and the tubes are of such a size and shape as to permit the nano-filtering of noxious immunological materials as described above. In a specific embodiment, the nano-tubes each have an effective diameter in the range from about 10 nanometers to about 30 nanometers. In another embodiment, the nano-tubes of said container have conical or pyramidal geometry.

In another embodiment of the present invention, two polymeric containers as described above are bonded together so as to form a closed capsule, wherein a portion of it contains a plurality of nano-tubes arranged in a predetermined manner.

The present invention contemplates a method of making a polymeric container having a plurality of nano-tubes arranged in a predetermined manner in a portion of it. The method comprises obtaining a container mold having a support structure. The support structure merely corresponds to the portion of the molded container to contain the aforementioned nano-tubes, and will generally define the inner volume of the container to be formed. A sacrificial layer is then supported by the support structure. A non-sacrificial moldable material is then discharged into the container mold, thereby covering said sacrificial layer. A nano-member array, as described above, is then impressed through the moldable material and into the sacrificial layer. The sacrificial layer is subsequently removed to reveal a plurality of nano-tubes. The tubes provided by this method will necessarily be arranged so that the inner volume of the container will be in fluid contact to the environment outside the container through the nano-tubes.

In another embodiment of the present invention, the aforementioned sacrificial layer comprises a soluble polymer. In yet another embodiment, the non-sacrificial material is in precursor form, and the method additionally comprises the step of setting the precursor material prior to the removal of the sacrificial layer. In yet another embodiment, the precursor material is selected from the group comprising thermoplastic solutions and spin-coated photocurable resins.

In general, the nano-member array utilized in the method for making a container can be any array as outlined above, and in one embodiment comprises an array of conical or pyramidal nano-members.

In an embodiment of the method of making a container, the support structure corresponds to an inner volume of the molded container of about 1 microliter.

The present invention contemplates a method for making a polymeric closed capsule containing a plurality of nano-tubes arranged so that the inner volume of the capsule is in fluid contact with the outer environment via the nano-tubes. The method comprises obtaining two polymeric containers having a plurality of nano-tubes arranged in a predetermined manner in a portion of at least one of the containers. The containers can be obtained by the method described above. The containers are then bonded together to form a capsule, wherein the capsule has an inner volume defined by inner volumes of the constituent polymeric containers. Bonding can be accomplished by any suitable means, including welding (ultrasonic, laser, or IR), lamination (adhesive tape, film thermal bonding), or resin-gas assisted bonding. In one embodiment, at least one of the containers has material deposited in it, such that the resultant closed container encloses the material. In another embodiment, the material comprises insulin-producing cells.

The present invention contemplates a micro-transfer mold comprising a polymeric plate containing a plurality of nano-tubes, whereby the nano-tubes are arranged in a predetermined manner, and a cavity plate arranged adjacent the polymeric plate, wherein the cavity plate contains a plurality of mold cavities dimensioned so as to provide nanoparticles. The cavity plate arranged adjacent the nano-tube plate may be obtained by any process capable of effecting micron or sub-micron cavities in a bulk material. Several embodiments of processes capable of effecting micron and sub-micron cavities in bulk material include, but are not limited to, differential etching, dry etching, photolithography, micro-injection molding, and embossing. These methods can effect mold cavities of varying sizes (<10 nm to >100 μm) and shapes (e.g. thin circular, oval, square or rectangular disk).

An embodiment of the micro-transfer mold comprises an additional layer arranged adjacent the polymeric plate, on the side of the plate opposite the cavity plate, wherein the additional layer is patterned so as to provide one or a series of material containers, or “transfer pots.” Such pots can, for instance, hold the moldable material to be urged through the nano-tubes into the mold cavities.

The patterned layer can be achieved by any means generally capable of imprinting a material in a predetermined manner so as to provide for such a transfer pot arrangement, such as photolithography. The present invention also contemplates the micro-transfer mold arrangement wherein the transfer pot arrangement is not a separate layer from the polymeric plate, but is achieved by forming the polymeric plate in a manner that provides such an arrangement. Such a plate itself defines the transfer pot or pots, or the volumes to contain the material to be urged through the nano-tubes of the micro-transfer mold. Such a plate can be manufactured in a manner analogous to that used to manufacture the polymeric container described above, wherein a portion of the container contains nano-tubes. In such a method as applied to achieving a molding apparatus, the volume defined by the container would be dimensioned for the purpose of forming a transfer pot.

The present invention contemplates a method of micro-transfer molding whereby a micro-transfer mold is obtained as outlined above and a moldable material is then urged through the nano-tubes into the mold cavities and allowed to set so as to form nanoparticles. In one embodiment the cavities of the cavity plate are partially filled with pre-deposited material prior to urging a moldable material through the nano-tubes into the mold cavities. The moldable material is then allowed to set so as to form microparticles containing said pre-deposited material. In a further embodiment, the pre-deposited material comprises any dry powder or granular material.

In one embodiment of the micro-transfer molding process, the additional step is added whereby the cavity plate containing the molded particles is packaged such that the cavity plate becomes the packaging carrier for the microparticles.

In another embodiment of the micro-transfer molding process a moldable material is urged through the nano-tubes into the mold cavities in an amount such that the cavities are only partially filled. The step of urging material through the nano-tubes is then repeated as necessary to fill the cavities, creating layered molded microparticles. In yet another embodiment, the successive iterations of partially filling the mold cavities utilize moldable material different from prior iterative step of partially filling the mold cavity, such that layered nanoparticles are formed wherein the layers comprise differing moldable materials.

### BRIEF DESCRIPTION OF THE DRAWINGS

FIG. 1A is an SEM micrograph of an array of conical nano-members produced by a differentially etching a fiber optic bundle using a buffered oxide etchant (BOE).

FIG. 1B is an SEM micrograph of an array of conical nano-members produced by anisotropic dry etching of silicon

FIG. 2A illustrates a diamond-tipped Atomic Force Microscopy (AFM) probe indenting a substrate to form a “master plate” for making a nano-member array.

FIG. 2B illustrates a material being cast onto the master plate of FIG. 2A.

FIG. 2C illustrates the nano-member array resulting from the casting of a material onto the master plate formed in FIG. 2A.

FIG. 2D is an SEM micrograph of a PMMA master plate, as depicted in FIG. 2A.

FIG. 2E is an SEM micrograph of a PDMS nano-member of a nano-member array as formed by a casting process as depicted in FIG. 2B.

FIG. 3A illustrates a step in the process of making a nano-tube plate utilizing a sacrificial layer, wherein the nano-member array is impressed through a non-sacrificial layer and into the sacrificial layer.

FIG. 3B illustrates the non-sacrificial layer of FIG. 3A after the nano-member array has been removed.

FIG. 3C illustrates the optional step of adding a patterned layer adjacent the non-sacrificial layer of FIG. 3B, whereby the patterned layer defines a volume or volumes for holding material.

FIG. 3D illustrates the resulting non-sacrificial and patterned layers of FIG. 3C subsequent to the removal of the sacrificial layer.

FIG. 4A illustrates a step in the process of making a nano-tube plate without the aid of a sacrificial layer, wherein a nano-member array is impressed into a non-sacrificial bulk material.

FIG. 4B illustrates another step in the process of making a nano-tube plate without the aid of a sacrificial layer, wherein the impressed bulk non-sacrificial layer of FIG. 4A has been cleaved along an x-y plane to reveal a substantial number of nano-tubes.

FIG. 4C is an SEM micrograph of a PDMS non-sacrificial bulk material that has been impressed by the array shown in FIG. 1A.

FIG. 5A illustrates a mold utilized in the method of making a polymeric container containing a plurality of nanotubes.

FIG. 5B illustrates the step in the method of making a polymeric container wherein a nano-member array is impressed through a non-sacrificial layer and into a sacrificial layer, wherein the sacrificial layer is supported on a supporting structure of the mold depicted in FIG. 5A.

FIG. 5C illustrates a polymeric container containing a plurality of nano-tubes resulting from the method depicted in FIG. 5B.

FIG. 5D illustrates a polymeric capsule containing a plurality of nano-tubes resulting from the method whereby two polymeric containers are bonded together.

FIG. 6 illustrates one embodiment of a micro-transfer mold of the present invention.

FIG. 7A illustrates an immunoprotective device comprising a nanofiltering capsule manufactured by the method disclosed herein.

FIG. 7B is a chart illustrating the typical size of materials related to an immunoprotective device.

### DETAILED DESCRIPTION OF THE INVENTION

It is to be understood that unless otherwise indicated, this invention is not limited to specific materials (e.g., specific polymers), processing conditions, manufacturing equipment, or the like, as such may vary. It is also to be understood that the terminology used herein is for the purpose of describing particular embodiments only and is not intended to be limiting.

It must be noted that, as used in the specifications and the appended claims, the singular “a,” “an,” and “the” include plural referents unless the context clearly dictates otherwise.

The prefix “micro” is used herein to refer to a dimension less than about 100 microns, but greater than about 1 micron.

The prefix “nano” is used herein to refer to a dimension less than about 100 microns, and includes dimension less than about 10 nanometers.

The term “nano-sprue” is used herein interchangeably with the term “nano-tube.”

The term “member” is used herein to refer to a projection that will result in forming a desired tube. Subsequently, the term “nano-member” is used herein to refer to a projection having an effective diameter on either end of less than about 100 microns, and includes projections having an effective diameter on either end of less than about 1 nanometer.

The term “nanoparticle” is used herein to refer to a three-dimensional solid structure whose height, width (diameter) or length is less than about 100 microns, and includes a three-dimensional solid structure whose height, width (diameter) or length is less than about 1 nanometer.

The term “plate” as used herein is intended to be inclusive of thin films. The thickness of a “plate” as used herein is meant to convey any thickness of material capable of substantially maintaining the structure of the nano-tubes present contained in the plate.

“Optional” or “optionally” means that the subsequently described circumstance may or may not occur, so that the description includes instances where the circumstance occurs and instances where it does not.

A novel approach to making polymeric plates containing a plurality of nano-tubes and articles of manufacture based on such a method is presented below. The approach can roughly be described as a method of embossing, and as such the “master” containing the embossing pattern is materially relevant to the resulting plate “embossed” with nano-tubes therein. The “master” for the purpose of this invention comprises an array of projections, or “nano-members” that define the arrangement and shape of the resulting nano-tubes.

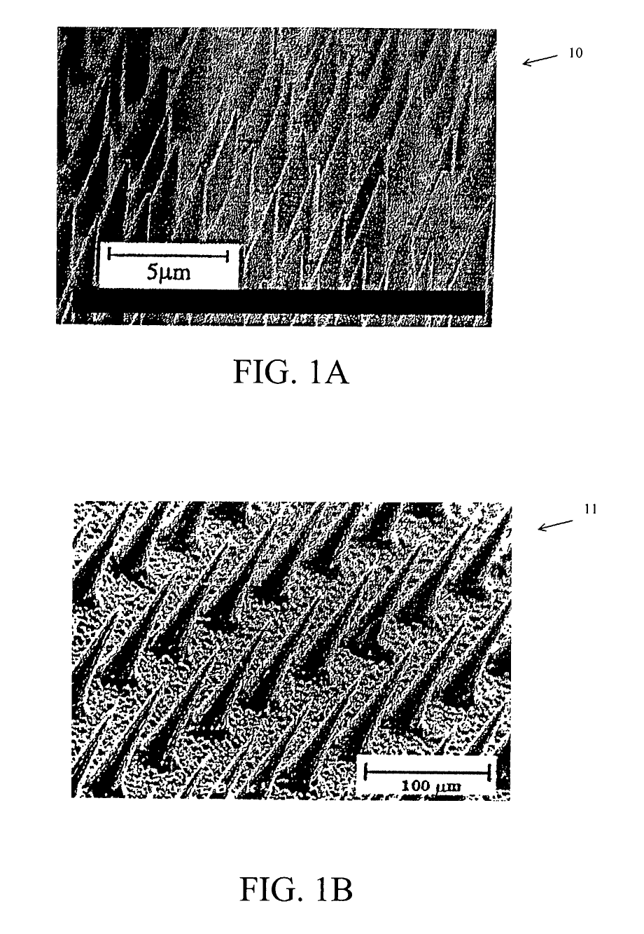

FIG. 1 is an SEM micrograph of one embodiment of a nano-member array. Nano-member array 10 is formed by differentially etching a fiber optic bundle, as generally described in T. H. Dam and P. Pantano, Review of Scientific Instrumentation, 70, 3982 (1999). Essentially, in the differential etching process, a buffered oxide etchant (BOE) etches the core and cladding layers of an optic fiber strand at different rates based on the gradient that exists in the level of dopant in those layers. A nano-member array formed as described therein may comprise conical nano-members with tip diameters less than about 20 nm and cone height in the range from about 1 micron to about 100 microns. Nano-member tip density may be as high as 10⁸/cm².

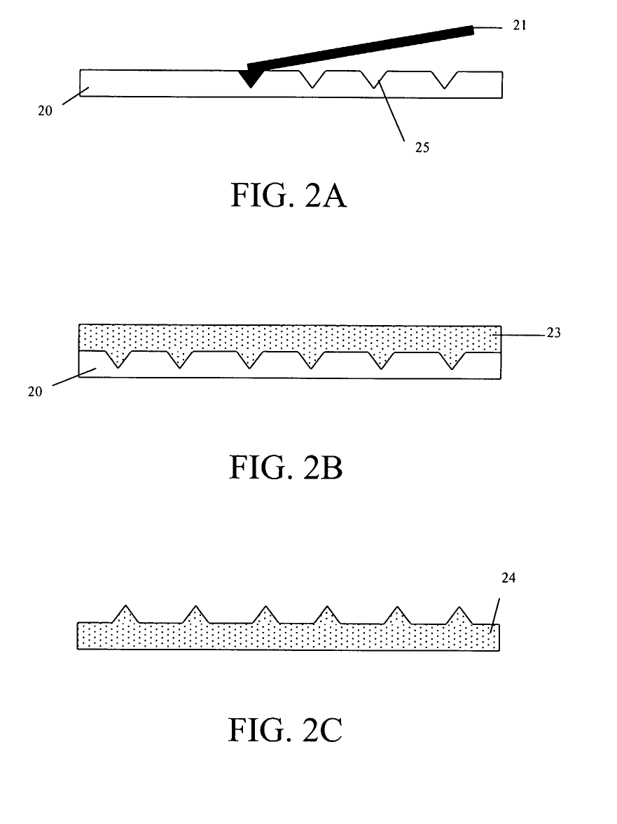

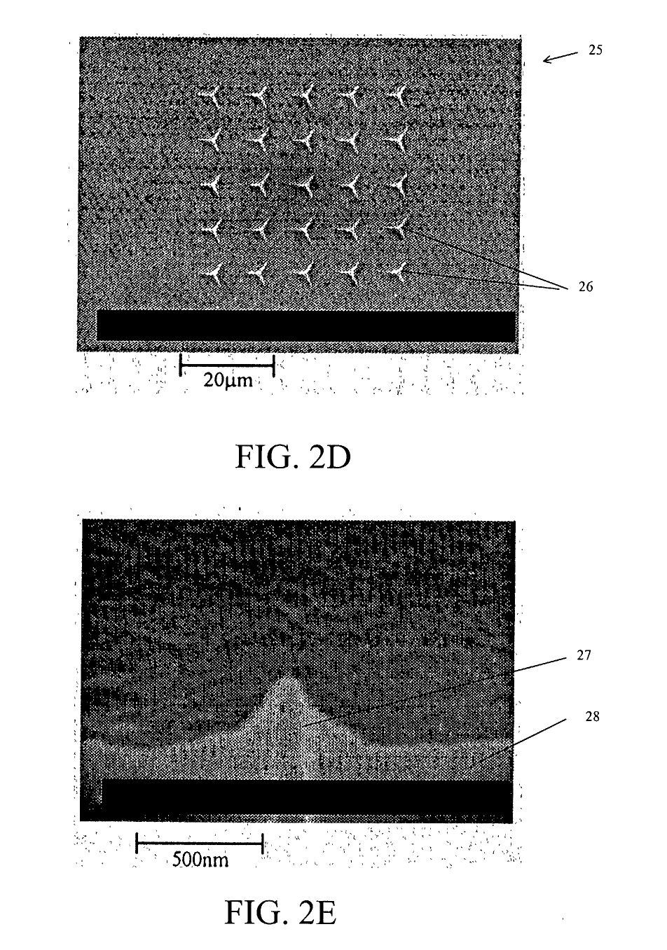

FIG. 2 is an SEM micrograph of another embodiment of a nano-member array utilized in the present invention. Nano-member array 11 may be produced by anisotropically etching silicon as described in S. Henry, D. V. McAllister, M. G. Allen and M. R. Prausnitz, Journal of Pharmaceutical Sciences, 87(8), 922 (1998). Essentially, anisotropic etching produces an array of conical members due to the different etching rates in the lateral and vertical directions of the silicon. An array produced via this method may result in conical members with heights on the order of 100 microns and tip diameters on the order of 1 micron.

FIG. 2 illustrates another method of making a nano-member array for use in the present invention. Generally, this embodiment comprises making a master plate to be used as a template for molding a nano-member array.

Specifically, FIG. 2A depicts the step of making master plate 20 by impressing a stylus instrument 21 into bulk polymeric material 22. A particular embodiment of stylus 21 comprises an Atomic Force Microscopy (AFM) probe tip. Bulk material 22 can comprise any polymeric material capable of receiving and maintaining an impression 25 and withstanding the subsequent casting process conditions involved in making a micro-member array. For example, bulk material 22 may comprise PMMA.

FIG. 2B depicts the step wherein the nano-member array material 23 is cast onto master plate 20 to form the array 24, depicted in FIG. 2C. Nano-member array material 23 can generally comprise any material suitable for casting and forming a nano-member array. For example, nano-member array material 23 may comprise PDMS.

FIG. 2D is an SEM micrograph of a particular embodiment 25 of master plate 20. Embodiment 25 was manufactured utilizing a 3-sided 90° pyramidal diamond AFM probe tip, with a radius of curvature of about 30 nanometers, using a force ranging from about 2500 to about 12,000 μN. Such an probe tip left impressions 26 in nano-member array material 22, comprising PMMA.

FIG. 2E is an SEM micrograph of a nano-member 27 resulting from casting nano-member array material 28 onto master plate embodiment 25. In this example, nano-member array material 28 comprises PDMS.

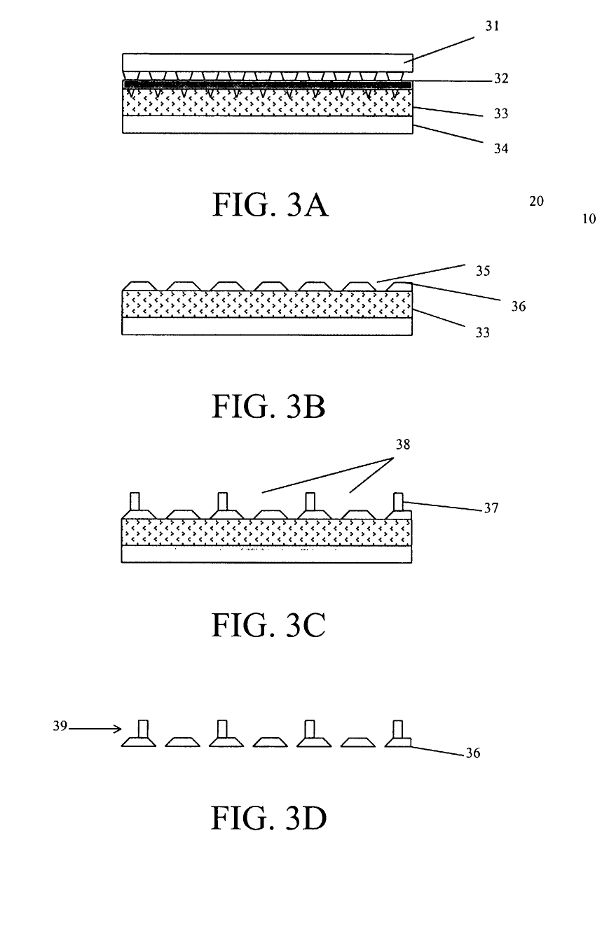

FIG. 3 generally depicts a method for making a nano-tube plate 36 utilizing a sacrificial layer 33 and including an optional patterned layer 39 arranged adjacent the nano-tube plate 36 to form material containers 38.

FIG. 3A depicts nano-member array 31 being impressed through non-sacrificial layer 32 and into sacrificial layer 33, which is adjacent support substrate 34. In one embodiment of the invention, non-sacrificial layer 32 is formed by spin-coating a thermoplastic polymer solution or photocurable resin precursor onto sacrificial layer 33. Particular embodiments of non-sacrificial layer 32 may include PDMS, any epoxy photoresist materials, HEMA, acrylics, PS, PC, and the like. Non-sacrificial layer 32 is then formed by partially or fully setting the coated polymer solution or precursor material by drying or UV curing.

Sacrificial layer 33 may be previously formed by coating a material onto support substrate 34 that is capable of being removed from non-transferrable layer 32. For instance, sacrificial layer 33 may comprise a soluble polymer material. In particular, sacrificial layer 33 may comprise a water soluble polymer. Examples of water soluble polymers include polyethylene oxide and poly (methacrylic acid, sodium salt). Support substrate 34 may be any suitable material capable of remaining dimensionally stable during processing. Particular emodiments of support substrate 34 include silicon, glass, or teflon.

FIG. 3B depicts the arrangement resulting from the removal of nano-member array 31, leaving nano-tube plate 36 affixed to sacrificial layer 33. Alternatively, nano-member array may be impressed through a previously set non-sacrificial layer 32 and removed to leave nano-tube plate 36 affixed to sacrificial layer 33. Examples, not intended to be limiting, of materials suitable for forming a pre-set non-sacrificial layer in which an array of nano-members is impressed, leaving a nano-tube plate, are: heated PMMA, partially cured PDMS, and partially cured epoxy photoresist materials.

The shape and dimensions of the nano-tubes such as nano-tube 35 are determined by, among other things, the size and shape of each nano-member of nano-member array 31, the depth that nano-member array 31 is impressed into sacrificial layer 33, and the material characteristics of non-sacrificial layer 32, which characteristics determine how well that layer retains the size and shape of the impressed nano-member array 31 upon its removal.

FIG. 3C depicts the addition of a patterned layer that forms material containers 38. Any method suitable for making meso-sized holes can be utilized to provide the patterned layer. Meso-sized hole wall 37 defines the material container. In one embodiment, the patterned layer is formed by photolithography.

FIG. 3D depicts the nano-tube plate 36 and patterned layer 39 after the sacrificial layer 33 has been removed. Removal of the sacrificial layer 33 can be achieved by any means suitable for removing the layer without damaging the nano-tube plate 36 or patterned layer 39. In one embodiment, the sacrificial layer 33 is a water soluble polymer which is subsequently removed by immersion in water.

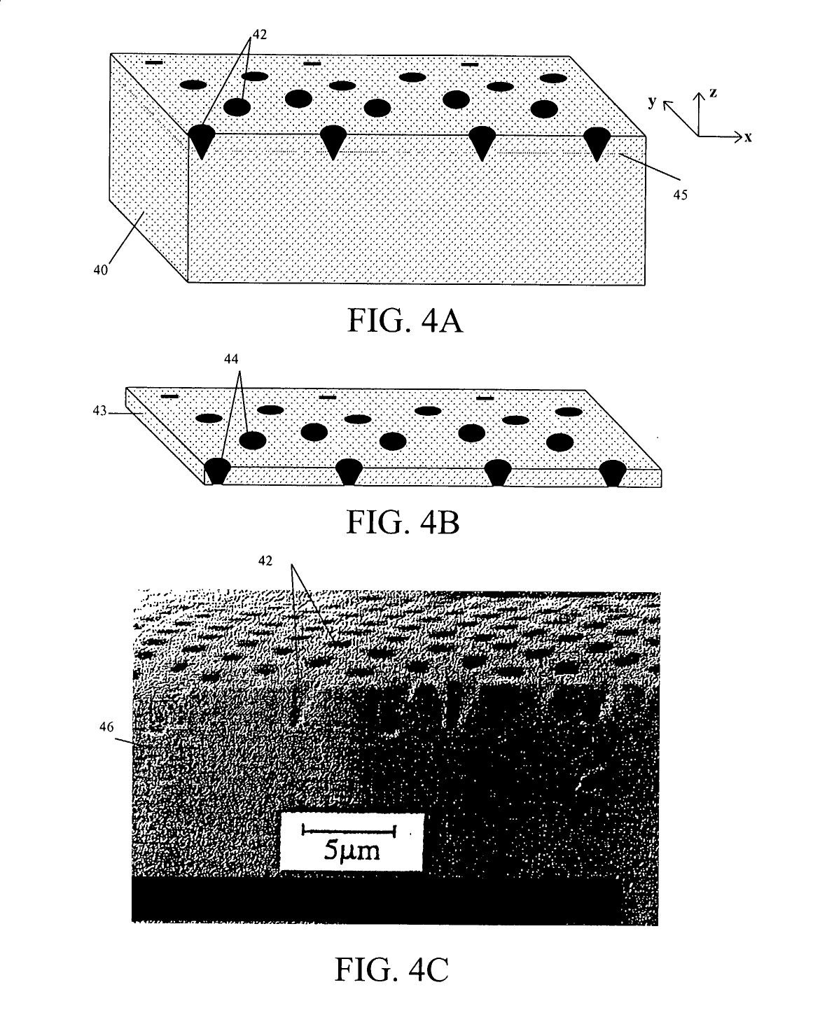

FIG. 4 generally depicts a method of manufacturing a polymeric nano-tube plate without the use of a sacrificial layer. FIG. 4A illustrates a bulk polymeric material 41 that has been impressed with a nano-member array, for instance nano-member array 10 depicted in FIG. 1A, leaving nano-impressions 42 in bulk polymeric material 41. FIG. 4B depicts the polymeric nano-tube plate 44 resulting when impressed bulk polymeric material 41 is cleaved in such a manner as to convert a substantial number of nano-impressions 42 into nano-tubes 44. Such a conversion can be generally achieved by cleaving bulk material 41 along the x-y plane 45 that intersects a substantial number of nano-impressions.

FIG. 4C is an SEM micrograph of a particular embodiment 46 of impressed bulk material 41 generally depicted in FIG. 4A. The particular embodiment 46 is PDMS that was manufactured by spin-coating a 10:1 mixture of silicone elastomer to curing agent onto a glass substrate and immersing nanomember array 10 into the spin-coated mixture film. The glass substrate was subsequently heated to about 70° C. to cure the PDMS. The nano-member array 10 was then removed from the cured PDMS. In general, cleaving can be accomplished by any mechanical means that will result in a nano-tube plate 43. One example includes guillotining impressed bulk polymeric material 41. Impressed bulk material 41 may optionally be cold or frozen to aid in a clean guillotining.

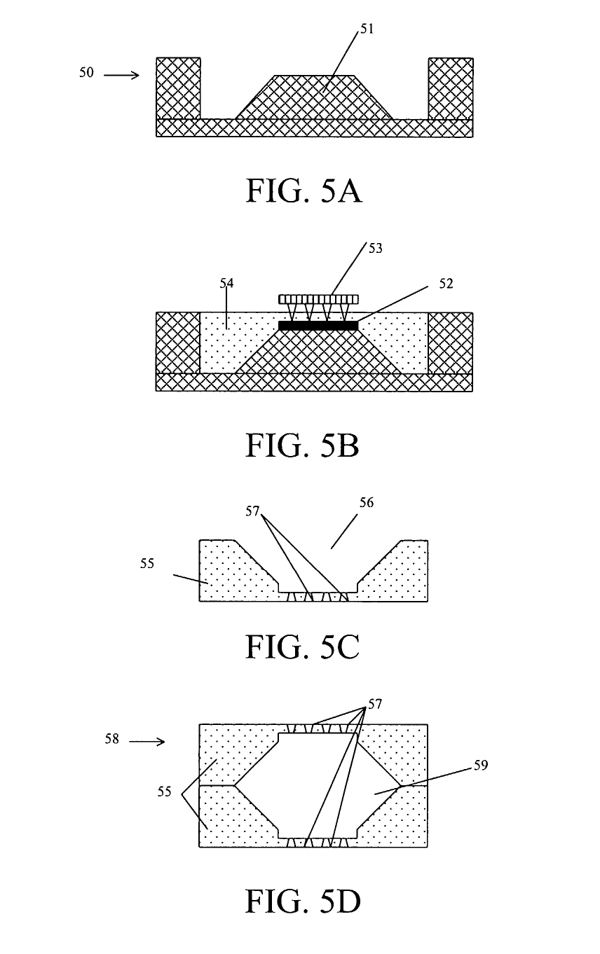

FIG. 5 generally depicts a method for making a polymeric container wherein a portion of the container wall contains a plurality of nano-tubes. The method is generally analogous to the method of making a polymeric plate previously described, and inferences may be drawn therefrom regarding suitable materials and methods. FIG. 5A depicts a mold 50 that defines the container 55 to be molded therein, and which generally includes a support structure 51 that defines an inner volume 56 to be encompassed by the container 55 and that acts as a base on which a sacrificial layer 52 can be coated or otherwise placed. FIG. 5B depicts the step wherein sacrificial layer 52 has been coated onto support structure 51 and wherein a non-sacrificial moldable material 54 has been charged into the mold 50. Furthermore, nano-member array 53 is impressed through non-sacrificial moldable material 54 and into the sacrificial layer 52. FIG. 5C depicts the finished container containing a plurality of nano-tubes 57 as defined by nano-member array 53. As is evident from FIG. 5C, the nano-tubes 57 are arranged so that inner volume 56 is in fluid connection through the nano-tubes 57 to the environment outside the container. Inner volume 56 is preferably about 1 microliter, but can be any volume suitable for the present invention, the limits of which volume are determined generally by the fabrication limitations of mold 50 and support structure 51. FIG. 5D depicts a polymeric capsule 58 manufactured by bonding two containers 55 and which has a plurality of nano-tubes 57 contained in its walls such that the enclosed inner volume 59 is in fluid connection to the environment outside capsule 58 only through nano-tubes 57. Examples of suitable bonding methods include welding (ultrasonic, laser, or IR), lamination (adhesive tape, film thermal bonding), or resin-gas assisted bonding.

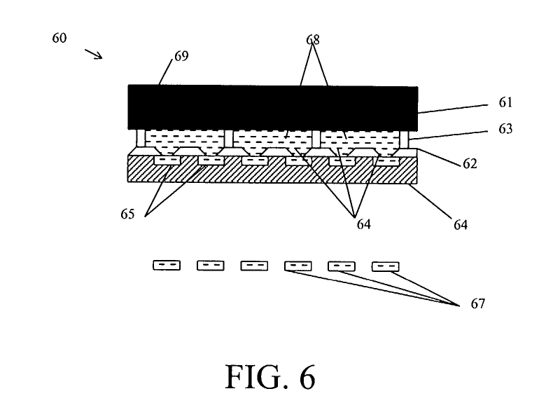

FIG. 6 generally depicts an apparatus and method for micro-transfer molding. Such a method is based on the well-known technique of transfer molding and permits a user to form microparticles 67 of differing shapes and sizes. The micro-transfer molding apparatus 60 is generally comprised of a polymeric nano-tube plate 62 with an adjacent patterned layer 63 defining material containers 68 obtained as outlined above. The molding apparatus is additionally comprised of a cavity plate 64 arranged adjacent the polymeric plate 62, wherein the cavity plate contains a plurality of mold cavities 65 dimensioned so as to provide nanoparticles 67. The cavity plate 64 arranged adjacent the polymeric nano-tube plate 62 may be obtained by any process capable of effecting micron or sub-micron cavities 65 in a bulk material. Several embodiments of processes capable of effecting micron and sub-micron cavities 65 in bulk material include, but are not limited to, differential etching, dry etching, photolithography, micro-injection molding, and embossing. These methods can effect mold cavities of varying sizes (<10 nm to >100 μm) and shapes (e.g. thin circular, oval, square or rectangular disk). Cavity plate 64 can be manufactured from any bulk or porous material suitable to have nano-cavities 65 defined therein and to withstand and permit subsequent processing conditions necessary to form nanoparticles 67. Examples of suitable bulk materials for cavity plate 64 include transparent material to permit any UV curing that may be necessary to form nanoparticles 67, such as glass, teflon, PDMS, and the like.

The method of micro-transfer molding generally depicted in FIG. 6 comprises charging a moldable nanoparticle material into material containers 68 and subsequently urging the moldable material through nano-tubes 66 by utilizing a plunger 69. Nano-tube plate 62 and cavity plate 64 are adjacent and in contact, and may optionally form a seal that would necessitate a venting tube arrangement. Certain materials and arrangements may necessitate the application of a vacuum to cavity plate 64 through a venting tube arrangement. The cavity plate 64 may then be separated form the nano-tube plate 62 for the purpose of further processing, as for example curing the nanoparticles 67.

It is to be understood that the present invention contemplates both batch and continuous processes for making nanoparticles 67 through the micro-transfer molding process as disclosed. The use of multiple mold cavity plates may help achieve a continuous process. In one embodiment of the present invention, cavity plate 64 itself is packaged with the nanoparticles 67 contained in cavities 65 to obtain an efficient means of producing and storing the nanoparticles.

In one embodiment of the present invention, the mold cavities 65 are filled with moldable material in iterative steps, wherein the moldable material partially fills the cavities 65 in each step, and wherein the moldable material may be different in different iterative steps, such that layered molded nanoparticles result. In another embodiment, mold cavities 65 have material pre-deposited in them prior to filling the cavities 65 with moldable material. In a particular embodiment, mold cavities 65 have pre-deposited therapeutic drug in them prior to the cavities being filled with a biodegradable polymer, such that the resulting nanoparticles are suitable for use as inhalation drug delivery particles.

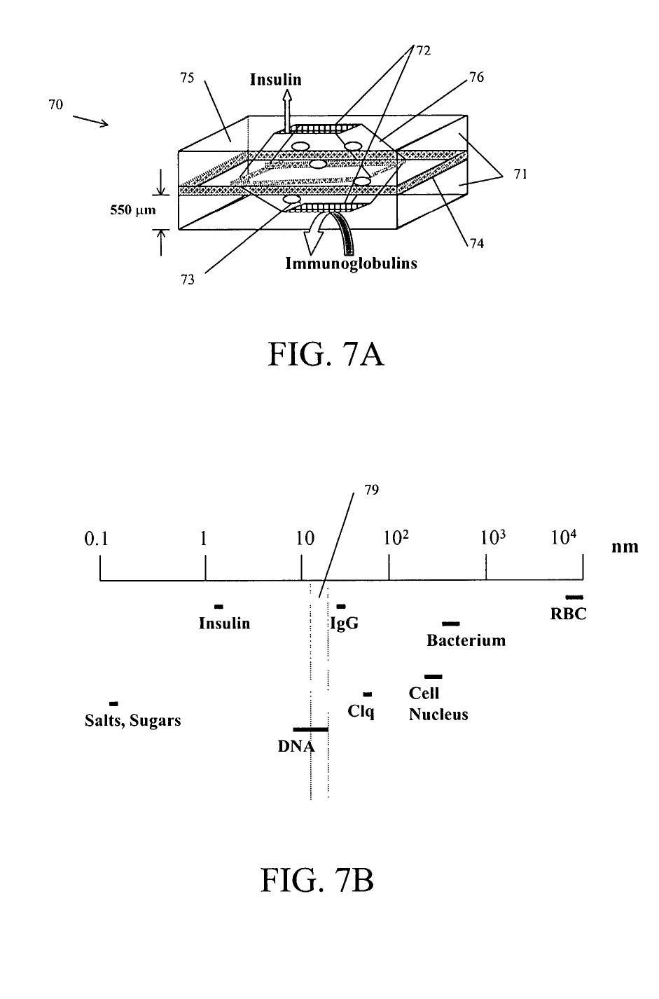

FIG. 7A depicts an immunoprotective device 70 as contemplated by the present invention. Such a device generally comprises a capsule containing a plurality of nano-tubes 72 contained in a portion of its walls 75, such that inner volume 76 is in fluid contact to the environment outside the capsule only through nano-tubes 72. Such a device 70 can be manufactured by the method outlined above for making a capsule from two molded containers 71 containing a plurality of nano-tubes 72 in a portion the container walls 75. Such containers 71 are bonded 74 to provide an inner volume 76. The effective diameter and shape of nano-tubes 72 are chosen so as to prevent particles of a larger effective diameter from passing-in effect acting as a screen. By screening particles larger than a certain effective diameter, immunoprotective device 70 can protect immunoprotective cells 73 contained in inner volume 76, such as microencapsulated insulin-producing cells, from noxious materials and prevent the escape of said immunoprotective cells 73 from the device 70.

FIG. 7B is a chart that depicts the sizes of materials relevant to an immunoprotective device 70. Size range 79, represents the range of nano-tube effective diameters necessary to provide an effective screening function for such a device. Noxious materials, generally those materials listed to the right of size range 79, are prevented from reaching immunoprotective cells 73, while beneficial materials, generally those materials listed to the left of size range 79, are permitted to freely pass through nano-tubes 72.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims, which are incorporated herein by reference.

I claim:

1. A polymeric plate containing a plurality of nano-tubes, said nano-tubes arranged in a predetermined manner.

2. The polymeric plate of claim 1, wherein at least one aperture of each of said nano-tubes has an effective diameter in the range from about 10 nanometers to about 100 nanometers.

3. The polymeric plate of claim 1, wherein said polymeric material is selected from the group consisting of photocurable and thermoplastic polymers.

4. The polymeric plate of claim 1 wherein said nano-tubes possess any geometry calculated to prevent substantially all material of a predetermined criterion to pass through said nano-tube while selectively allowing substantially all other material to pass, whereby said polymeric plate acts as a nanofilter.

5. The polymeric plate of claim 1 wherein said nano-tubes possess geometry selected from the group consisting of conical and pyramidal geometry.

6. A method of making a polymeric plate containing a plurality of nano-tubes comprising the steps:
obtaining a starting material arrangement comprising:
a support substrate;
a sacrificial layer supported by said substrate; and
a non-sacrificial layer on said sacrificial layer;
impressing an array of nano-members through said non-sacrificial layer and into said sacrificial layer; and
removing said sacrificial layer.

7. The method of claim 6 wherein said support substrate comprises a material selected from the group consisting of silicon, glass, teflon, and any other polymer capable of substantially maintaining dimensional stability upon increased heating.

8. The method of claim 6 wherein said sacrificial layer comprises a soluble polymer.

9. The method of claim 6 wherein said non-sacrificial layer in said starting material is in precursor form and wherein the method additionally comprises setting said precursor prior to removal of said sacrificial layer.

10. The method of claim 9 wherein said precursor form material is selected from the group consisting of thermoplastic solutions and spin-coated photocurable resins.

11. The method of claim 6 wherein said nano-member array comprises an arrangement of projections, said projections having effective diameters on either of their ends ranging from about 10 nanometers to about 100 nanometers.

12. The method of claim 6 wherein said nano-members possess any geometry calculated to prevent substantially all material of a predetermined criterion to pass through said nano-tube while selectively allowing substantially all other material to pass, whereby said polymeric plate acts as a nanofilter.

13. The method of claim 6 wherein said nano-members possess geometry selected from the group consisting of conical and pyramidal geometry.

14. The method according to claim 6 wherein said nano-member array is a material selected from the group consisting of a fiber optic bundle that has been differentially etched, silicon that has been anisotropically etched, and a polymer tip array that has been formed using a master plate containing nano-scale surface projections.

15. The method of claim 6 additionally comprising providing a patterned layer over said non-sacrificial layer so as to provide a material container in association with said non-sacrificial layer.

16. The method of claim 15 wherein the patterned layer is formed by photolithography.

17. A method of making a polymeric plate containing a plurality of nano-tubes comprising the steps:
obtaining a starting material arrangement of bulk material precursor;
impressing an array of nano-members into said bulk material precursor;
setting said bulk material precursor;
removing said array of nano-members; and
cleaving said bulk material precursor so as to expose a series of apertures.

18. The method of claim 17 wherein said bulk material precursor is selected from the group consisting of partially cured thermoset and heated thermoplastic polymers.

19. A polymeric container defining an inner volume wherein a portion of said container's walls contain a plurality of nano-tubes, said nano-tubes arranged in a predetermined manner and positioned so as to place said inner volume in fluid contact with an outer environment.

20. The polymeric container of claim 19 wherein said inner volume is less than about 1 microliter.

21. The polymeric container of claim 19, wherein at least one aperture of each of said nano-tubes has an effective diameter in the range from about 10 nanometers to about 100 nanometers.

22. The polymeric container of claim 19 wherein said nano-tubes possess geometry selected from the group consisting of conical and pyramidal geometry.

23. A polymeric nano-filtering capsule comprising an inner volume enclosed by a polymeric surface, wherein a portion of said surface contains a plurality of nano-tubes, said inner volume in fluid contact with an environment outside said polymeric walls only through said nano-tubes.

24. The polymeric nano-filtering capsule of claim 23 wherein said inner volume is less than about 1 microliter.

25. A method of making a polymeric container defining an inner volume wherein a portion of said container's walls contain a plurality of nano-tubes, said nano-tubes arranged in a predetermined manner and positioned so as to place said inner volume in fluid contact with an outer environment, said method comprising the steps:
obtaining a starting material arrangement comprising:
a container mold having a support structure, wherein said support structure corresponds to a portion of a container wherein a plurality of nano-tubes are to be prearranged;
a sacrificial layer supported by said support structure;
discharging a non-sacrificial material into said container mold, wherein said sacrificial material layer is covered;
impressing an array of nano-members through said non-sacrificial layer and into said sacrificial layer;
removing said sacrificial layer.

26. The method of claim 25 wherein said sacrificial layer comprises a soluble polymer.

27. The method of claim 25 wherein said non-sacrificial material is in precursor form and wherein the method additionally comprises setting said precursor prior to removal of said sacrificial layer.

28. The method of claim 27 wherein said precursor form material is selected from the group consisting of thermoplastic solutions and spin-coated photocurable resins.

29. The method of claim 25 wherein said nano-member array comprises an arrangement of projections, said projections having effective diameters on either of their ends ranging from about 10 nanometers to about 100 nanometers.

30. The method of claim 25 wherein said support structure corresponds to an inner volume of said container less than about 1 microliter.

31. The method of claim 25 wherein said nano-members possess geometry selected from the group consisting of conical and pyramidal geometry.

32. A method of making a polymeric nanofiltering capsule wherein an inner volume is enclosed by a polymeric surface and a portion of said surface contains a plurality of nano-tubes, said inner volume in fluid contact with an environment outside said polymeric walls only through said nano-tubes comprising the steps:
obtaining two polymeric containers whose surfaces each define an inner volume, at least one of which surfaces contains a plurality of nano-tubes arranged in a predetermined manner; and
bonding together said containers to form a capsule, wherein said capsule has an inner volume defined by a surface defined by the bonded constituent surfaces of said polymeric containers.

33. The method of claim 32 wherein said capsule inner volume is at least about 600 nanoliters.

34. A micro-transfer mold comprising:
a polymeric plate containing a plurality of nano-tubes, said nano-tubes arranged in a predetermined manner; and
a cavity plate containing a plurality of mold cavities arranged adjacent said non-sacrificial layer, wherein said mold cavities are dimensioned so as to form nanoparticles.

35. The micro-transfer mold of claim 34, additionally comprising a patterned layer arranged adjacent said polymeric plate to provide a material container, or transfer pot, in association with said polymeric plate, said patterned layer positioned on the side of said polymeric plate opposite said cavity plate.

36. The micro-transfer mold of claim 34 wherein the patterned layer is formed by photolithography.

37. A micro-transfer mold comprising:
a polymeric container defining an inner volume wherein a portion of said container's walls contain a plurality of nano-tubes, said nano-tubes arranged in a predetermined manner and positioned so as to place said inner volume in fluid contact with an outer environment; and
a cavity plate containing a plurality of mold cavities arranged adjacent said non-sacrificial layer, wherein said mold cavities are dimensioned so as to form nanoparticles.

38. A method of micro-transfer molding comprising the steps:
obtaining a micro-transfer mold;
urging a moldable material through a plurality of nano-tubes in said micro-transfer mold and into mold cavities of said micro-tranfer mold; and
allowing said moldable material to set so as to form molded nanoparticles.

39. A method according to claim 38 comprising the additional step of packaging the cavity plate containing molded nanoparticles present in the mold cavities, said cavity plate becoming the carrier for said nanoparticles.

40. The nanoparticles produced by the method of claim 38

41. A method of micro-transfer molding comprising the steps:
obtaining a micro-transfer mold wherein a plurality of said micro-transfer mold's mold cavities are partially filled with pre-deposited material;
urging a moldable material through a plurality of nano-tubes in said micro-transfer mold and into mold cavities of said micro-tranfer mold; and
allowing said moldable material to set so as to form molded nanoparticles that contain pre-deposited material.

42. A method according to claim 41 wherein said pre-deposited material comprises material selected from the group consisting of dry powder and granular materials.

43. The nanoparticles produced by the method of claim 41.

44. A method of micro-transfer molding comprising the steps:
obtaining a micro-transfer mold;
urging a moldable material through a plurality of nano-tubes in said micro-transfer mold and into mold cavities of said micro-tranfer mold such that the mold cavity is partially filled; and
repeatedly urging moldable material into said mold cavities as necessary so as to form layered molded nanoparticles.

45. A method according to claim 44 wherein the step of urging moldable material into said mold's partially filled mold cavities utilizes moldable material different from the moldable material utilized in a prior iteration of urging moldable material into said mold cavities so that layered nanoparticles are formed, whereby the nanoparticle layers comprise differing moldable materials.

46. The nanoparticles produced by the method of claim 44.

47. The nanoparticles produced by the method of claim 45.
